(19) United States
(12) Patent Application Publication
(10) Pub. No.: US 2010/0261275 A1
(43) Pub. Date: Oct. 14, 2010

(54) Production of Recombinant Interferon Proteins

(76) Inventors: Yves Durocher, Montreal (CA); Martin Loignon, Kirkland (CA); Brian Cass, Chateauguay (CA)

Correspondence Address: NATIONAL RESEARCH COUNCIL OF CANADA, 1200 Montreal Road, Building M-58 Room EG-12, OTTAWA, ON, K1A 0R6, CA

(21) Appl. No.: 12/746,787

(22) PCT Filed: Dec. 10, 2008
(86) PCT No.: PCT/CA08/02167

§ 371 Date: Jun. 8, 2010

Related U.S. Application Data
(60) Provisional application No. 60/996,901, filed on Dec. 10, 2007.

(51) Int. Cl. C12N 5/071; C07K 14/555

(52) U.S. Cl. 435/369; 530/416; 530/395

(57) ABSTRACT

A method of purifying a recombinant interferon protein involves providing an aqueous mixture of the recombinant protein and contaminating proteins; precipitating the contaminating proteins from the aqueous mixture at a pH in a range of from 0.5 to 6; separating the aqueous mixture from the precipitated contaminating proteins; and, eluting the separated aqueous mixture through a cation exchange column using a mobile phase with a salt or pH gradient, the gradient being from lower salt concentration or pH to higher salt concentration or pH, to produce a recombinant interferon protein fraction separated from other components of the aqueous mixture. The method provides for the recovery of recombinant interferon proteins in better yield and purity.

### CROSS-REFERENCE TO RELATED APPLICATIONS

This application claims the benefit of U.S. Provisional Patent Application U.S. Ser. No. 60/996,901 filed Dec. 10, 2007, the entire contents of which is herein incorporated by reference.

### FIELD OF THE INVENTION

The present invention relates to biotechnology, in particular to methods for producing and purifying recombinant interferon proteins.

### BACKGROUND OF THE INVENTION

Interferons are cytokines with major therapeutic applications based on their antiviral, antiproliferative, and immunomodulatory activities. The interferon alpha2b (IFN-α2b) is the predominant subvariant detected in human genomic DNA [1]. Some of the many diseases treated with IFN-α2b, alone or in combination, include type B [2] and C hepatitis [3], several cancers such as melanoma [4-6], Kaposi's sarcoma [7], chronic myeloid lymphoma [8,9], and angioblastoma [10]. In the particular case of hepatitis C, a disease affecting over 170 million individuals worldwide, the combination of IFN-α and the viral inhibitor ribavirin has become the standard treatment [11-13]. The rising incidence of certain cancers and viral hepatitis [14,15], in addition to ongoing investigations of novel therapeutic applications [16] are increasing the needs for human recombinant IFN-α2b.

Human recombinant IFN-α2b used in the clinic is synthesized in bacterial systems. When E. coli are grown in optimal conditions, a few grams (3 to 5) of recombinant human IFN-α per liter of culture can be produced [17-19]. Bacterially produced recombinant human IFN-α2b is misfolded and therefore requires refolding into its native conformation. Once purified and refolded, the recoveries are typically lower than 20% [17,18]. This refolding process also often results in loss of specific activity. In addition, bacterially produced recombinant human IFN-α2b lacks the post-translational O-glycosylation present on the naturally synthesized protein. This non-glycosylated form of human recombinant IFN-α2b has a shorter serum half-life than the glycosylated form [20]. The chemical conjugation of polyethylene glycol (PEG) molecules to the core peptide (peggylation) to create a mixture of positional isomers has improved the pharmacodynamics of IFN-α2b by increasing the serum half-life [21]. However, the peggylation of IFN-α2b has been reported to reduce its biological activity [22]. It has also been proposed that the size of PEG molecules and sites of attachment differentially interfere with the interaction and binding of IFN-α2b to its receptor [23]. Another common problem associated with the use of peggylated IFN-α is the formation of neutralizing antibodies. Antibody formation against peggylated IFN-α in HCV-infected patients has been associated with treatment failure [24,25]. It is not known yet if the appearance of these antibodies is the result of the presence of PEG moieties or from contaminating partially unfolded IFN species.

Human and other mammalian cells are expression systems of choice for the production of secreted recombinant proteins such as antibodies, sometimes yielding up to hundreds of milligram to gram quantities of purified product per liter of culture [26-28]. However, the volumetric productivity of human cells for given proteins such as cytokines (i.e. IFN-α2b) is often lower by several orders of magnitude. Originally, IFN-α for therapeutic use was purified from the human lymphoblastoid Namalwa cell line. Increase in volumetric productivity was achieved following induction with Sendai virus. Functional studies showed that the biological properties of IFN-α produced by Namalwa cells are altered by the glycosylation inhibitor tunicamycin, highlighting the importance of glycosylation for optimal IFN-α activity [29]. Despite the production of an IFN-α with high biological activity, Namalwa cells were abandoned due to a limited productivity unable to satisfy an ever-growing demand. Other systems have been tested for the production of INF-α2b. Avian eggs have also been assayed for the production of human recombinant IFN-α2b [30,31], although the glycosylation pattern significantly differs from IFN-α2b produced by human peripheral blood leucocytes. Glycosylated IFN-α2b can be produced in decent yields in insect cells, but glycosylation is of the potentially immunogenic high-mannose type and also lacks sialylation [32]. These limitations suggest that mammalian cells are preferable hosts for the production of fully glycosylated IFN-α2b. Chinese hamster ovary (CHO) cells have been used for the production of various human recombinant interferons. Glycosylated and biologically active mouse IFN-α [33] can be produced in CHO cells. Similarly, Rossman et al have reported the production of 120 μg/mL of IFN-α2b in a glutamine synthase-amplified mouse myeloma cell line NSO [34]. This is the highest level of glycosylated recombinant human IFN-α2b produced in a mammalian system reported to date. In vitro, the biological activity of NS0-produced IFN-α2b is very similar to that produced by Namalwa cells.

Purification of recombinant proteins (r-proteins) (e.g. recombinant IFN-α2b) often pose problems, especially when they contain no affinity tags. Purification of r-proteins often represents the major cost for biotherapeutic manufacturing. Recombinant proteins are often purified using a combination of ion-exchange, hydrophobic, reverse-phase and size-exclusion chromatography (among other). Each chromatographic step has a significant impact on the overall recovery of the product. For example, a 3-steps process with 60% recovery at each step will have a final yield of 21.6%, representing a net product loss of 78.4%. It is thus important to minimize the number of steps and to ensure highest yield as possible at every step.

There remains a need in the art for an effective method of purifying recombinant proteins (e.g. recombinant IFN-α2b) in high yield and purity.

### SUMMARY OF THE INVENTION

There is provided a method of purifying a recombinant interferon protein comprising: providing an aqueous mixture of the recombinant protein and contaminating proteins; precipitating the contaminating proteins from the aqueous mixture at a pH in a range of from 0.5 to 6; separating the aqueous mixture from the precipitated contaminating proteins; and, eluting the separated aqueous mixture through a cation exchange column using a mobile phase with a salt or pH gradient, the gradient being from lower salt concentration or pH to higher salt concentration or pH, to produce a recombinant interferon protein fraction separated from other components of the aqueous mixture.

With the exception of presently disclosed stable HEK293 cell clone (namely D9) capable of producing hundreds of milligrams of human recombinant IFN-α2b, recombinant cytokines are typically produced in much lower quantities in mammalian expression systems. The difficulty of producing high quantities of cytokines and other proteins such as VEGF165b emphasizes the importance of developing purification processes allowing high purity and recovery. Volumetric production of recombinant interferon protein from the D9 clone can reproducibly exceed 250 mg/L in batch culture, and can even exceed 300 mg/L, and can remain stable in culture in the absence of selection for a long time, for example nine months or more. Cells are grown in an aqueous mixture, for example a cell culture medium. Preferably, the cells are grown in serum-free culture medium. Even when the production is performed in serum-free medium, significant amounts of cell-derived contaminants accumulate in the culture over time.

It has now been found that following acidification (below about pH 6) of the aqueous mixture, an important precipitate forms that is mostly composed on contaminating proteins, while surprisingly the recombinant protein remains soluble and not denatured even down to a pH of about 0.5. In preferred embodiments, precipitating the contaminating proteins is accomplished at a pH in a range of from about 2 to about 5, for example from about 3.6 to 3.8. Acidification may be achieved using any suitable acid, preferably an organic acid. Some examples of suitable acids include citric acid, acetic acid, hydrochloric acid, phosphoric acid, etc. This acid precipitation may also be very useful as a viral inactivation step as many enveloped viruses are inactivated this way (it is widely used in the manufacturing process of biotherapeutics).

Following acidification of the aqueous mixture, the mixture is preferably clarified, for example by centrifugation or filtration. The mixture is loaded onto a cation-exchange column. Cation exchange columns comprise a stationary phase having anionic moieties, for example sulfonic acid, quaternary amine and carboxylic acid moieties. Preferably, the cation exchange column comprises a stationary phase with sulfonic acid moieties. Elution is done using a salt or a pH gradient in the mobile phase, thereby allowing elution of the recombinant protein as a single peak. The purity of the recombinant protein in this peak can be 95% or greater (e.g. 98% or greater) with an overall yield of 70% or more, or 75% or more, e.g. 75-85%. For example, the pH gradient may be from pH 3.5 to pH 6, or the salt gradient from 80 mM to 2 M. pH may be adjusted with any suitable buffering system, for example mixture of free acid and sodium salts of: citric acid, acetic acid, formic acid, phosphoric acid or a mixture thereof. The salt for the salt gradient is preferably a physiological acceptable salt, for example sodium chloride, potassium chloride, ammonium acetate, ammonium sulfate, ammonium formate or any mixture thereof.

In an embodiment of the present invention, a non-amplified IFN-producing clone derived from the HEK293 mammalian cell line that produces hundreds of milligrams of human glycosylated INF-α2b per liter of serum-free media has been made. A rapid and efficient method for its purification was also developed. The volumetric production of IFN-α2b largely and reproducibly exceeds 200 mg/L in batch culture and remains stable in the absence of selection for more than nine months in culture. The IFN-α2b is purified by single-step cation-exchange chromatography following media acidification and clarification. This rapid procedure yields 98% pure IFN-α2b with a recovery greater than 75%. Purified IFN-α2b migrates on SDS-PAGE as two species, a major 22 kDa band and a minor 19 kDa band. N-terminal sequences of both forms are identical and correspond to the expected mature protein. Purified IFN-α2b forms stable non-covalent dimers at neutral pH with an apparent molecular weight of 44,000 Da as determined by size-exclusion chromatography. The presence of intramolecular disulfide bridges is evidenced by the fact that non-reduced IFN-α2b has a greater electrophoretic mobility than the reduced form. Treatment of purified IFN-α2b with neuraminidase followed by O-glycosidase both increases electrophoretic mobility, indicating the presence sialytated O-linked glycan. A detailed analysis of glycosylation by mass spectroscopy identifies disialylated and monosialylated forms as the major constituents of purified IFN-α2b. Electron transfer dissociation (ETD) shows that the glycans are linked to the expected threonine at position 129. Other minor glycosylated forms and non-sialylated species are also detected, similar to IFN-α2b produced naturally by lymphocytes. Further, the HEK293-produced IFN-α2b is biologically active as shown with a gene reporter assay. Together these results demonstrate that cost-effective production and purification of glycosylated IFN-α2b from human cells can be achieved.

A simple, scalable and high yield method for high level purification of IFN has been provided.

Further features of the invention will be described or will become apparent in the course of the following detailed description.

### BRIEF DESCRIPTION OF THE DRAWINGS

In order that the invention may be more clearly understood, embodiments thereof will now be described in detail by way of example, with reference to the accompanying drawings, in which:

FIG. 1. Expression plasmid encodes a codon-optimized sequence of human IFN-α2b. A) The pYD7-IFNα2b expression plasmid was used to generate D9 clone. (Amp) ampicillin, (Blast) blasticidin, (CMV) cytomegalovirus promoter, (enh MLP) adenovirus major late promoter, (IFN-α2b) codon-optimized sequence for human IFN-α2b gene, (pA) polyadenylation sequence, (pMBlori) bacterial origin of replication, (Puro) puromycin, (OriP) Epstein-Bar virus origin of replication, (SV40pA) simian virus 40 polyadenylation sequence, (TPL) adenovirus tripartite leader. B) Amino acid sequence of human IFN-α2b (SEQ ID NO: 1). Signal peptide is underlined. The two intramolecular disulfide bridges are C1-C98 and C29-C138. The glycan-linked threonine (Thr107) is underscored.

FIG. 2. Kinetics of cell growth and IFN-α2b production from D9 clone in fed-batch culture. D9 cells were seeded at a cell density of 0.25×106 cells per mL, fed with 0.1% TN1 the next days and sampled every day. A) Coomassie-stained SDS-PAGE gel of the production media (20 μL) collected daily. B) Cell counts, viability and IFNα-2b were measured daily.

FIG. 3. Purification of IFN-α2b by cation-exchange chromatography. A) A typical chromatographic profile of a purification of HEK293-produced IFN-α2b from a 400 mL fed-batch culture is illustrated. Solid line shows the 280 nM absorbance profile. Dotted line shows pH variations. IFN-α2b elutes in a single peak between 1000 and 1200 mL. B) Coomassie-stained SDS-PAGE gels of 20 μL samples collected at different steps of production and purification of IFN-α2b. 1—crude harvest. 2—Precipitate (equivalent to 200 μL of harvest volume). 3—Clarified harvest. 4—Flow through SO₃⁻ column. 5—Wash SO₃⁻ column. 6—Elution peak SO₃⁻ column. 7—Desalted IFN-α2b in PBS.

FIG. 4. pH-dependent precipitation of IFN-α2b from 8 days D9 harvest (250 ml production).

FIG. 5. Purified IFN-α2b form dimers at neutral pH independent of intermolecular cystine formation. Following a desalting step in neutral PBS, purified IFN-α2b was analysed for dimer formation. A) Twenty mg of purified IFN-α2b were analysed on a Superdex™ 75 equilibrated with PBS pH 7.0. The arrows and numbers above indicate the elution volumes of molecular weight standards eluted in the same conditions. Purified IFN-α2b elutes in the same volume as ovalbumin a 44 kDa globular protein. B) Coomassie-stained SDS-PAGE gels of samples (20 μL) of each of the 10 fractions (4 mL) collected between elution volume 40-80 mL. C) Coomassie-stained SDS-PAGE gels of reduced and non-reduced IFN-α2b from HEK293 cells.

FIG. 6. HEK293-produced human IFN-α2b is sialylated and O-glycosylated. IFN-α2b was deglycosylated as described in material and methods. 1-10 μg of purified D9-produced IFN-α2b. 2-10 μg of purified D9-produced IFN-α2b digested with neuraminidase. 3-10 μg of purified D9-produced IFN-α2b digested with O-glycosidase. 4-10 μg of purified E. coli-produced IFN-α2b.

FIG. 7. ESI-MS analysis of the intact IFN-α2b glycoprotein. A) ESI mass spectrum exhibiting the glycoform profiles associated with each charge state of the protein and B) the glycoprotein molecule weight profile reconstructed from the mass spectrum in panel a. The most intense peak at 20,213 Da appears to be composed of the mature peptide chain plus a single core type-1 disialylated glycan (Hex1 HexNAc1 SA2).

FIG. 8. CID and ETD analysis of the tryptic glycopeptides from IFN-α2b. A) CID-MS/MS spectrum of the triply protonated ion at m/z 1426.8 corresponding to the disialylated glycopeptide of T84-112. The spectrum is dominated by the sequential neutral loss of the glycan components from the doubly protonated glycopeptide ion. The principal b and y fragment ions arising from fragmentation of the peptide backbone are indicated in the spectrum as are the compositions the glycan oxonium ions observed m/z 494.9, 657.0 and 948.0, respectively. The sequence of the peptide is provided in the inset. B) CID-MS/MS spectrum of the triply protonated ion at m/z 1340.8 corresponding to the monosialylated glycopeptide of T84-112. Note that the neutral loss corresponding to a second sialic acid is missing from this spectrum as is the corresponding oxonium ion at m/z 948.0. C) ETD MS/MS spectrum of the triply protonated, monosialylated T84-112 glycopeptide at m/z 1340.8. The higher m/z region of the ETD spectrum contained the most informative fragment ions and is presented here. The c ion series indicated in the spectrum clearly identified the site of O-linkage as Threonine 107 of the mature protein.

FIG. 9. HEK293-produced human IFN-α2b is biologically active. The biological activity of HEK293-produced human IFN-α2b was assayed with a gene reporter assay and compared to E. coli-produced human recombinant IFN-α2b as described in material and methods. The activity of the secreted alkaline phosphatase is plotted against the concentration of IFN-α2b produced in the two hosts. Each point represents the average±SEM of 3 experiments performed in triplicate.

### DESCRIPTION OF PREFERRED EMBODIMENTS

### Materials and Methods

### Materials

Expression plasmid was purified with a maxi-prep plasmid purification kit (Qiagen, Mississauga, ON, Canada). F17 serum-free culture media and blasticidin were obtained from Invitrogen (Carlsbad, Calif.). Pluronic™ F68 and glutamine were from Sigma-Aldrich (St. Louis, Mo.) and Tryptone N1 from Organotechnie (La Courneuve, France). Reagents for IFN-α2b purification and electrophoresis include anhydrous citric acid and tri-Na citrate (EMD Chemicals Inc, Darmstadt, Germany) 0.45 mm filtering units (Millipore, Bedford, Mass.), NaCl (Sigma-Aldrich, St. Louis, Mo.), Fractogel™ SO₃⁻ (M) (Merck KGaA, Darmstadt, Germany), Econo-Pac® 10 columns (Bio-Rad Laboratories), Bradford Reagent (Biorad, Hercules, Calif.) 2 μm filters (Pall Corp, Ann Arbor, Mich.), NuPAGE Bis Tris 4-12% gradient gels, MES 20× buffer (Invitrogen, Carlsbad, Calif.), and Coomassie stain (Sigma-Aldrich, St. Louis, Mo.). Trypsin (Promega, Madison Wis.), neuraminidase, dithiothreitol, iodoacetamide and guanidine HCl (Sigma-Aldrich, St. Louis, Mo.), O-glycosidase (Roche), Tris HCl, (Bio-Rad, Mississauga, ON), high purity acetonitrile, formic acid and ammonium bicarbonate (VWR International, Montreal, QC) and Centricon™ 3,000 MWL centrifugal filters (Millipore, Bedford, Mass.) were used for glycosylation analysis. The IFN-αantibody and ELISA kit are from PBL Biomedical Laboratories (New Brunswick, N.J., USA) and bacterially produced IFN-α2b from Cell Sciences Inc (Norwood, Mass., USA). pNifty2-56K-SEAP plasmid is from Invivogen (San Diego, USA).

### IFN-α2b Expression Plasmid

The IFN-α2b gene was synthesized with human-optimized codons (Geneart AG, Regensburg, Germany) according to the GenBank accession no. AY255838. The synthetic cDNA was inserted as a BamHI/EcoRI fragment downstream of the cytomegalovirus (CMV) promoter into the pYD7 expression plasmid. This plasmid is a derivative of the previously described pTT vector [35] encoding the original functional elements in addition to a blasticidin resistance cassette.

### Engineering of an IFN-α2b Expressing HEK293-6E Stable Clone and Fed-Batch Production

A HEK293 cell line constitutively expressing the EBNA1 protein of EBV (clone 6E) was used to generate IFN-producing clones. HEK293-6E and IFN-producing clone are grown in suspension in serum-free F17 culture media supplemented with 1% Pluronic™ F68. Cultures are grown at 37° C. and 5% CO₂ under constant agitation (120 rpm). HEK293-6E were transfected as previously described [35] with Pvul-linearized pYD7/IFN-α2b and selected in the presence of 2 μg/mL of blasticidin. The blasticidin resistant cells were next seeded into 96 well plates at 1 cell/well without blasticidin. After 3-4 weeks, emerging clones were expanded (in the absence of blasticidin) and tested for IFN-α2b expression by dot blot. The selection of IFN-producing clones was based on the levels of IFN-α2b expression and growth properties of the clones. The highest producers were amplified as suspension cultures and tested for IFN-α2b accumulation over 4 days of culture. One clone, identified as D9, was selected because it is stably producing high IFN-α2b levels while maintaining a high growth rate (doubling time of 24 hours). The D9 clone was deposited at the International Depositary Authority of Canada, National Microbiology Laboratory, Public Health Agency of Canada, 1015 Arlington Street, Winnipeg, Manitoba, Canada, R3E 3R2, under accession no. 021208/03 on Dec. 5, 2008, the contents of which are herein incorporated by reference. For IFN-α2b production, cells are seeded at a density of 0.25×106 cells/mL in F17 antibiotic-free media in shaker flasks. Twenty-four hours post seeding, the cultures are fed with 0.5% peptones [35,46] and the cells are grown for an additional 7-8 days. Optional addition of glucose (5-25 mM) and glutamine (1-5 mM) is performed 4 days post seeding.

### Purification of IFN-α2b

The culture medium of a fed-batch culture is collected by centrifugation at 1000 g for 10 min. The supernatant is then acidified to pH 3.6-3.8 with 1 M citric acid. Acidification causes the formation of a precipitate which is removed by centrifugation. The clarified supernatant is then filtered on 0.45 mm filtering unit. Purification of IFN-α2b from the filtered supernatant is performed on an ÄKTA Explorer™ system (GE Healthcare, Baie D′Urfé, QC, Canada). The supernatant is loaded at a flow rate of 10 mL/min on a Fractogel™ SO₃⁻ cation exchange column, previously equilibrated with 0.1 M Tri-Na citrate buffer pH 3.5 containing 0.35 M NaCl. Following a wash with 2 column volumes of the equilibration buffer, the IFN-α2b is then eluted with a pH gradient. The pH of the mobile phase is increased from pH 3.5 to pH 6.0 with 0.1 M Tri-Na citrate buffer pH 6.0, plus 0.35 M NaCl. The fractions containing IFN-α2b are pooled. An additional desalting step is performed on Econo-Pac® 10 columns according to the manufacturer's specifications. For the determination of glycosylation by enzymatic digestion the purified IFN-α2b, is desalted in 0.1 M NH₄HCO₃ buffer pH 5 and lyophilized, whereas for bioassays the purified IFN-α2b is desalted in PBS and sterile filtered.

### Quantification and Purity of IFN-α2b

IFN-α2b recovered from the SO₃⁻ column was quantified by measuring absorption at 280 nm in a spectrophotometer, with a Nanodrop™ ND-1000 (Fisher Scientific, Montreal, QC, Canada), with a Bradford assay and by ELISA according to the manufacturer's protocol. The concentration in the harvest was measured with ELISA and used to calculate the percent recovery. To assess the purity level of IFN-α2b, 3 mg were analyzed by SDS-PAGE followed by Coomassie staining.

### N-Terminal Sequencing and Enzymatic Determination of Glycosylation of Purified IFN-α2b

As HEK293-produced IFN-α2b migrates as two bands on SDS-PAGE, N-terminal amino acid sequences from both bands were obtained by automated sequencing performed at our sequencing facility. Enzymatic treatments with neuraminidase and O-glycosidase were performed to remove sialic acid and O-linked sugars respectively. Sequential digestions were performed in 50 mM phosphate buffer pH 5.0 on 100 μg of purified/desalted IFN-α2b. Removal of sialic acid was done with 0.5 IU of neuraminidase for 1 h at 37° C. followed by addition of 15 mU of O-glycosidase. Non-glycosylated recombinant IFN-α2b from E. coli and, glycosylated and deglycosylated HEK293-produced IFN-α2b were resolved on SDS-PAGE in parallel to compare migration profiles. Migration profiles of glycosylated and deglycosylated HEK293-produced IFN-α2b were compared to non-glycosylated IFN-α2b produced in E. coli.

### Analysis of Intact IFN-α2b by Mass Spectrometry

The protein solution (about 1 μg/μL in PBS buffer) was desalted by filtration on a 3,000 MWL Centricon™ filter and diluted to its original concentration with deionized water. The solution was adjusted to 20% acetonitrile, 0.2% formic acid just prior to infusion at 1 μL/min into the electrospray interface of a Q-TOF 2 hybrid quadrupole time-of-flight mass spectrometer (Waters, Milford, Mass.). The mass spectrometer was set to acquire one spectrum every 2 seconds over the mass range, m/z 800-2600. The protein molecular weight profile was generated from the mass spectrum using MaxEnt™ (Waters).

Sequence Analysis of the Tryptic Glycopeptides from Purified IFN-α2b

Purified IFN-α2b was reduced, alkylated and digested with trypsin according to standard protocols. In summary, approximately 100 μg of the protein was dissolved in 1M Tris HCl, 6M guanidine HCl, pH 7.5 containing 2 mM dithiothreitol (DTT) and incubated at 500° C. for 1 hour. The reduced cysteines were converted to carboxyamidomethyl derivatives using 10-fold excess of iodoacetamide over DTT. The protein solution was then concentrated on a 3,000 MWL Centricon™ and diluted to 100 μL using 50 mM ammonium bicarbonate. This process was repeated a second time. Trypsin (5 μg) was added to the sample, which was then incubated overnight at 370° C.

The tryptic digest was analyzed and fractionated by LC-MS using an Agilent™ 1100 HPLC system coupled with the Q-TOF2 mass spectrometer. Approximately, 60 μg of the protein digest was injected onto a 4.6 mm×250 cm Jupiter, 5 μm, 300 Å, C18 column (Phenomenex, Torrance, Calif.) and resolved using the following gradient conditions: 5% to 60% acetonitrile, 0.2% formic acid in 45 minutes, increasing to 95% after 50 minutes (1 mL/min flow rate). Approximately, 60 μL/min of the HPLC eluate was directed to the mass spectrometer while the remainder was collected in 1 minute fractions. The Q-TOF2 mass spectrometer was set to acquire 1 spectrum per second (m/z 150-2000) whilst cycling between a low and high offset voltage within the collision cell (10 V and 35V, respectively). This enabled the simultaneous detection of intact peptide and glycopeptide ions in the higher m/z regions (low offset mode) as well as the unique glycan oxonium ions in the lower regions of the spectrum (high offset mode). By screening the fractions in this manner it was possible to determine that only two of them (fractions 25-26 and 26-27 minutes, respectively) contained glycopeptides.

Glycopeptides were interrogated by collision induced dissociation (CID) to determine their amino acid sequence and glycan composition and by electron transfer dissociation (ETD) to identify the site of linkage. ETD preserves delicate modifications intact during the fragmentation process and is ideal for identifying the linkage sites of O-glycans [47-49]. The glycopeptide-containing fractions were infused at 1 μL/min into the electrospray ionization source of a LTQ XL linear ion trap (Thermo Fisher Scientific) capable of performing ETD. The CID collision voltage was adjusted for optimum production of peptide fragment ions from the multiply charge glycopeptide precursor ions (typically 25-30 V). ETD was performed using fluoranthene as the anionic reagent and with supplementary activation enabled. The optimal ETD reaction time for these glycopeptides was 350 msec.

### Biological Activity

A SEAP reporter gene assay based on expression plasmid containing an IFN-inducible promoter (pNiFty2) was used to assess the biological activity of glycosylated HEK293-produced IFN-α2b in comparison to non-glycosylated IFN-α2b. This experiment was performed as previously reported [50]. Briefly, HEK293 cells were transfected with the pNiFty2 reporter plasmid, which encodes the secreted embryonic alkaline phosphatase (SEAP) under the control of the human ISG56 promoter. Transfected cells were plated in 96 well plates at a cell density of 105 cells/mL and stimulated, 24 h post transfection, with IFN-α2b at the indicated concentrations. Following an additional 48 h period of incubation, the supernatants were collected and assayed for SEAP activity. The specific hydrolysis of paranitrophenyl phosphate (pNPP) was measured as a function of time to determine SEAP activity induced with IFN treatments, according to our previously described procedure [50]. SEAP activity is expressed the increase in absorbance at 410 nm over time.

### Results:

### Generation of a Stable IFN-expressing HEK293 Cell Clone and Production of IFN-α2b in Fed-Batch Cultures

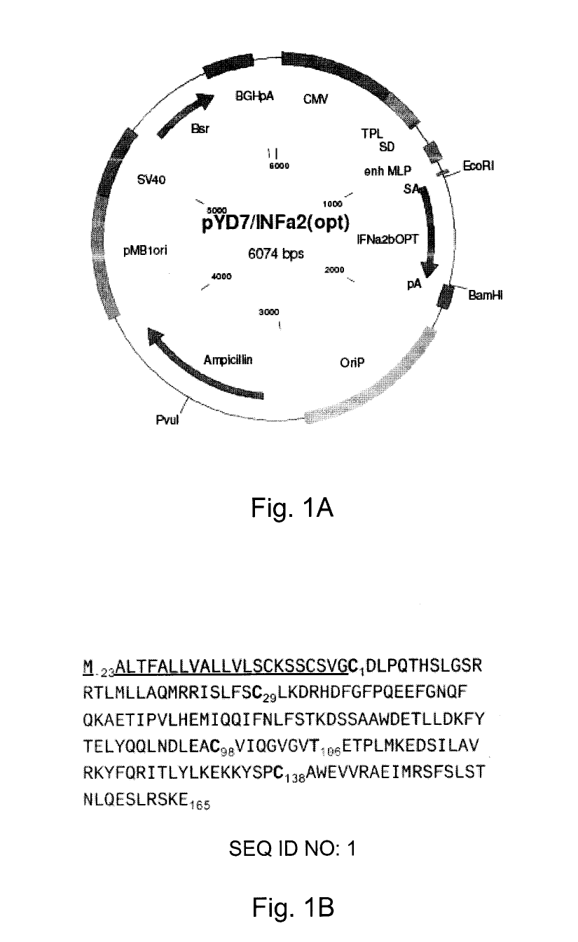

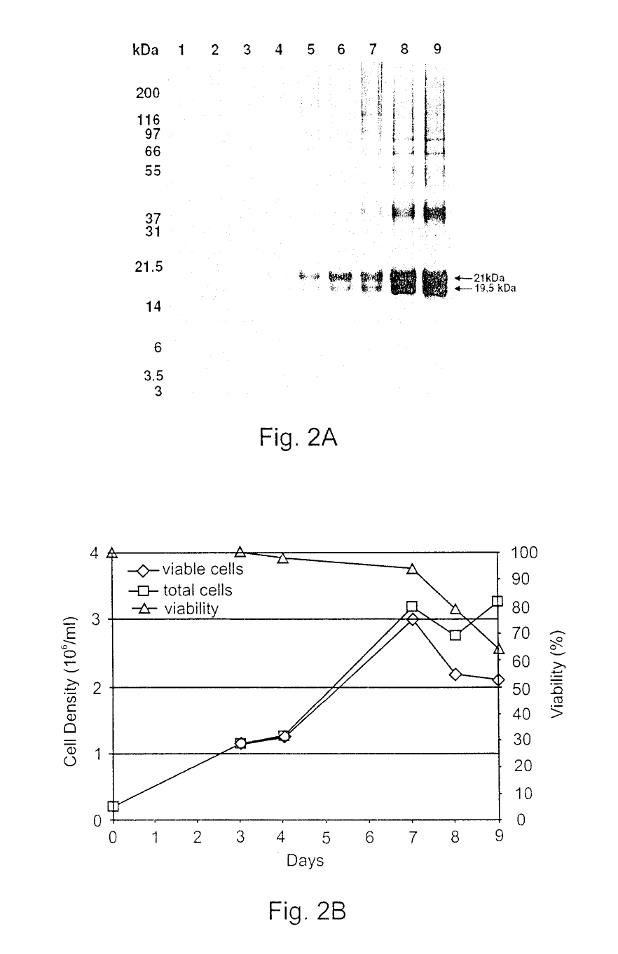

The expression plasmid pYD7 encoding the human IFN-α2b gene codon-optimized for expression in human cells (FIG. 1A) is derived from the previously described pTT vector [35]. The signal peptide sequence, cysteine residues involved in intramolecular cystine formation, and the threonine of the consensus sequence for O-glycosylation of human IFN-α2b are highlighted (FIG. 1B). The calculated molecular weight of the mature core protein (a.a. 24-188) of IFN-α2b is 19,269 Da. In order to generate IFNα2b-producing cells, HEK293 were transfected with linearized pYD7-IFNα2b and selected in the presence of blasticidin. The D9 clone, which stably produces IFN-α2b was isolated as described in materials and methods. The production of IFN-α2b with the D9 clone was performed in fed-batch culture. A Coomassie-stained gel of daily samples from the culture media sampled daily for a period of 9 days shows that the levels of IFN-α2b plateau at 8 days (FIG. 2A), time at which cell-derived contaminating proteins begin to accumulate significantly. This is also the period of culture corresponding to a decline in cell number and viability (FIG. 2B). Therefore, fed-batch productions were harvested at this point. It is noteworthy that, early during production, HEK293-produced IFN-α2b migrates with an apparent molecular weight of 2 kDa greater than its predicted mass calculated from the amino acid sequence (19.3 kDa), while at around day 4, a less abundant band of about 19.5 kDa appears.

### Purification of Recombinant IFN-α2b by Cation Exchange Chromatography and Analysis by Gel Filtration and SDS-PAGE

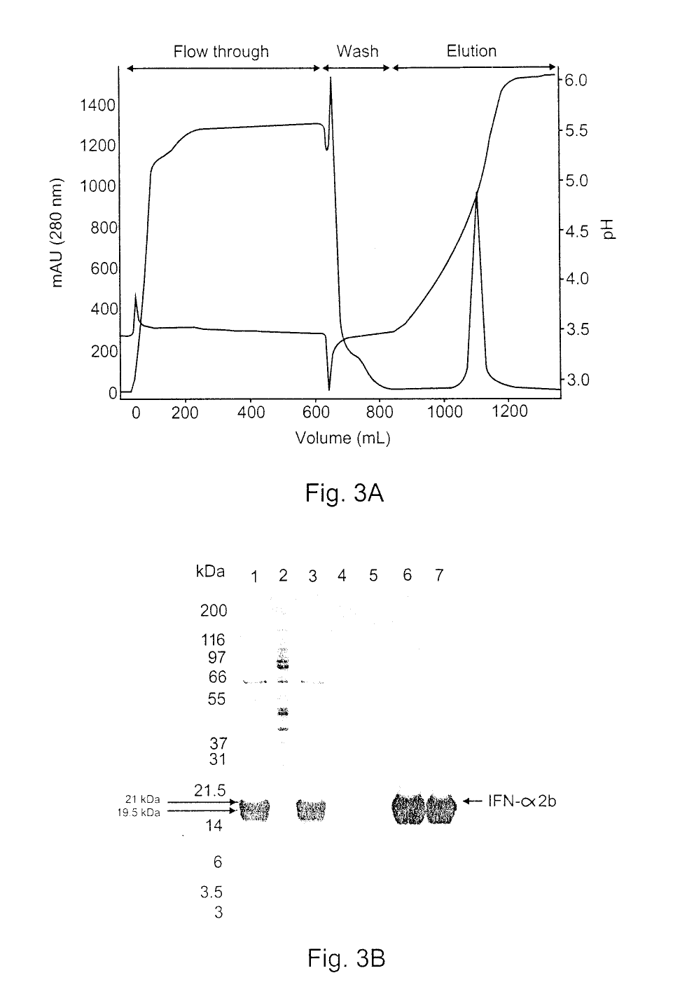

At the end of the production phase, the IFN-α2b is purified as described in material and methods. The IFN-α2b elutes in a single peak at pH 4.5-4.6 from the cation exchange column (FIG. 3A). The electrophoretic profiles of proteins contained in the harvest, the acid precipitate, the clarified harvest and eluted fractions, are shown on a Coomassie-stained gel (FIG. 3B). The low level of IFN-α2b in the acid precipitate highlights the efficacy of acidification step to selectively remove protein contaminants. The absence of IFN-α2b in the flow through and in the wash suggests that IFN-α2b strongly binds to the SO₃⁻ column. According to a conservative estimate performed by densitometric analysis of the SDS-PAGE resolved purified material, the purity of IFN-α2b exceeds 98% after the SO₃⁻ column and the final desalting step.

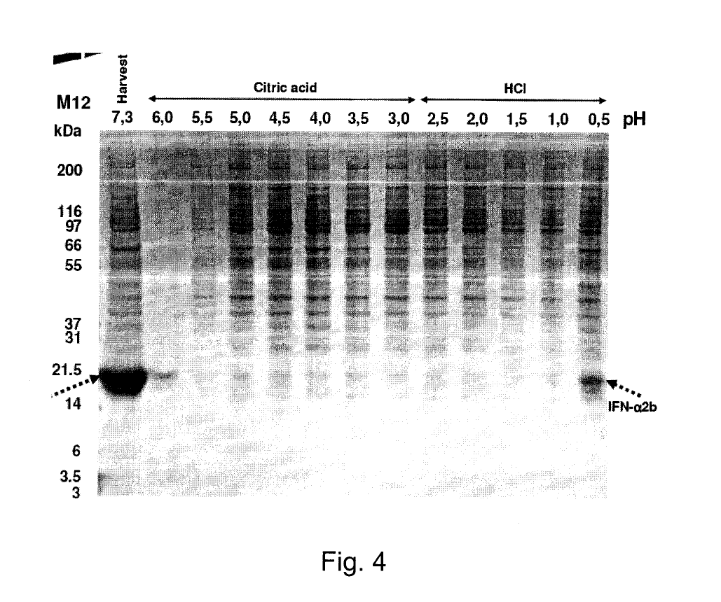

The pH-dependence of precipitation of IFN-α2b is shown in FIG. 4. The pH from a D9 cells harvest was lowered incrementally by 0.5 pH units using citric acid or HCl. The resulting precipitates were washed with PBS and resuspended in 100 μL of 1×LDS buffer per mL of harvest. Two mL equivalents of each precipitate were ran in parallel with the harvest (the pH of which was 7.3). No significant precipitate was detectable at pH about 6.0. Also, IFN-α2b does not significantly precipitate at pH above 0.5.

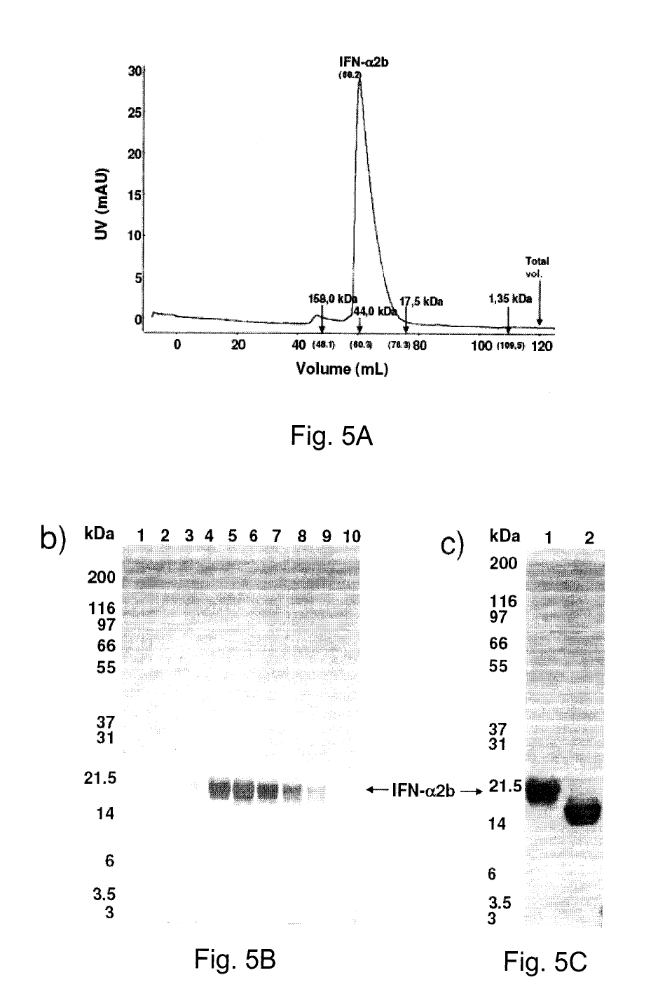

Following desalting in PBS, purified IFN-α2b was loaded on a Superdex™ 75 gel filtration column. The peak elution volume is almost identical to that of ovalbumin, a 44 kDa protein, indicating that HEK293-produced IFN-α2b forms dimers at neutral pH (FIG. 5A). A Coomassie-stained SDS-PAGE gel of IFN-containing fractions shows species with different electrophoretic mobilities (FIG. 5B), reflecting some glycosylation heterogeneity in the purified material. Under reducing conditions, purified IFN-α2b migrates as a major band of approximately 21 kDa, whereas under non-reducing conditions, IFN-α2b migrates with an apparent molecular weight of about 17 kDa, a greater electrophoretic mobility typical of the presence of intramolecular disulfide bridges (FIG. 5C). The absence of dimers (i.e. about 42 kDa band) in non-reducing conditions, indicate that the formation of dimers is independent of intermolecular disulfide bridges.

The D9 Clone Produces Hundreds of Milligrams of IFN-a2b Per Liter of Culture that are Efficiently Recovered

IFN-α2b in the crude harvests of fed-batch cultures was quantified by ELISA. The average concentration from two independent productions is 237±11 mg/L, with a maximum of 316 mg/L when extra glucose and glutamine are added during production (Table 1). IFN-α2b recovered from the SO₃⁻ column measured by ELISA correlated well with measures obtained with a Bradford assay and by absorbance at 280 nm using IFN-α2b molar extinction coefficient. The concentrations of IFN-α2b measured by ELISA in the harvest and in the recovered fraction from the SO₃⁻ column were used to determine the recovery. The mean concentration of IFN-α2b shows that between 70 and 80% of the IFN-α2b produced could be recovered, for two independent productions for each condition (Table 1). These results are comparable in terms of volumetric productivity and recovery to some productions of non-glycosylated IFN-α2b performed in E. coli and in the methyltrophic yeast Pichia pastoris (Table 2).

TABLE 1
Quantification and Recovery of IFN-α2b
from Two Production Schemes

| Production | IFN-α2b mg/L (ELISA) | | |
|---|---|---|---|
| Scheme | Crude harvest | SO₃⁻ column | Average Recovery (%) |
| 1 feeding | 237.1 ± 11 | 185.1 ± 3 | 79.5 |
| 2 feedings | 301 ± 25 | 216.1 ± 11 | 71.8 |

TABLE 2
Overview of Human Recombinant IFN-α2b Production in Prokaryotic and
Eukaryotic Systems

| | | Recovery | | | | Activity | |
|---|---|---|---|---|---|---|---|
| Host | mg/L | mg/L | % | Purity % | Glycosylation | IU/mg* | Ref. |

Prokaryotic

| | | | | | | | |
|---|---|---|---|---|---|---|---|
| E. coli | 5200 | 3000 | 58 | ND | No | 3 × 10⁹ | [19] |
| E. coli | 4000 | 300 | 7.5 | ND | No | 2.5 × 10⁸ | [17] |
| E. coli | 2500 | 600 | 12 | 100 | No | ND | [18] |
| S. lividans | 0.01 | ND | ND | ND | No | 0.4 × 10⁴ | [51] |

Eukaryotic

| | | | | | | | |
|---|---|---|---|---|---|---|---|
| Pichia pastoris | 450 | 298 | 66.2 | >95 | ND | 1.9 × 10⁹ | [40] |
| BY-2 tobacco | 0.02 | ND | ND | ND | No | ND | [52] |
| SFP insect | ND | ND | ND | ND | Partial | 2.3 × 10⁸ | [32] |
| | | | | | No sialylation | | |
| NS0 mouse | 120 | ND | ND | ND | Yes | 2 × 10⁸ | [34] |

*IFN-α2b activity has been determined by inhibition of viral replication. Different viruses and hosts were used.

### IFN-α2b Produced in HEK293 is β-Glycosylated, Highly Sialylated and Biologically Active

One of the major therapeutic interests for producing IFN-α2b in mammalian cells is to generate a fully active and glycosylated protein. The apparent molecular weight of the major 21 kDa product observed on SDS-PAGE suggests either that IFN-α2b produced in HEK293 undergoes post-translational modifications or less likely, that the signal peptide is incorrectly processed. There is also a less abundant product of around 19.5 kDa on SDS-PAGE. In order to ascertain that the signal peptide is accurately processed, N-terminal sequencing was performed on both products. The sequences obtained were identical and read C-D-L-P-Q-T, as expected for a correctly processed signal peptide.

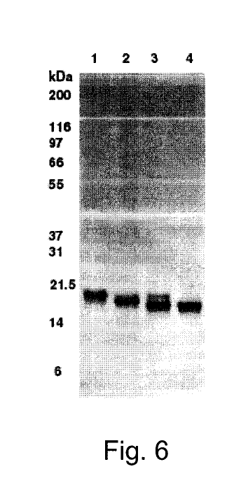

We next determined whether IFN produced in HEK293 is, O-glycosylated [36] and sialylated as previously reported for IFN-α2b produced by human peripheral blood leucocytes [37]. We performed a sequential digestion of purified IFN-α2b with neuraminidase and O-glycosidase to respectively remove sialic acid residues and O-linked saccharides. Each digestion successively increase the electrophoretic mobility of purified IFN-α2b to generate a deglycosylated product that migrates as fast as non-glycosylated recombinant IFN-α2b produced E. coli (FIG. 6), demonstrating that IFN-α2b produced in HEK293 cells is O-glycosylated and sialylated. Note here the quasi absence of the lower about 19.5 kDa product in the lane containing the non-digested IFN. We found that the majority of this product is lost during the purification process, as most of it remains bound to the column (data not shown). A minor band with lower electrophoretic mobility was still visible after glycosidases treatment, suggesting that this species might be fucosylated.

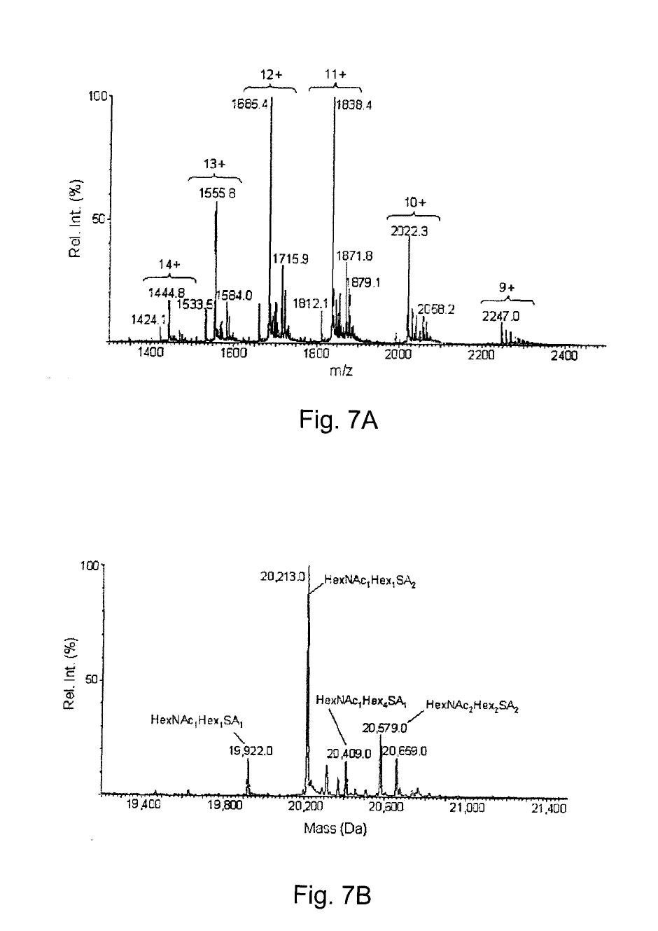

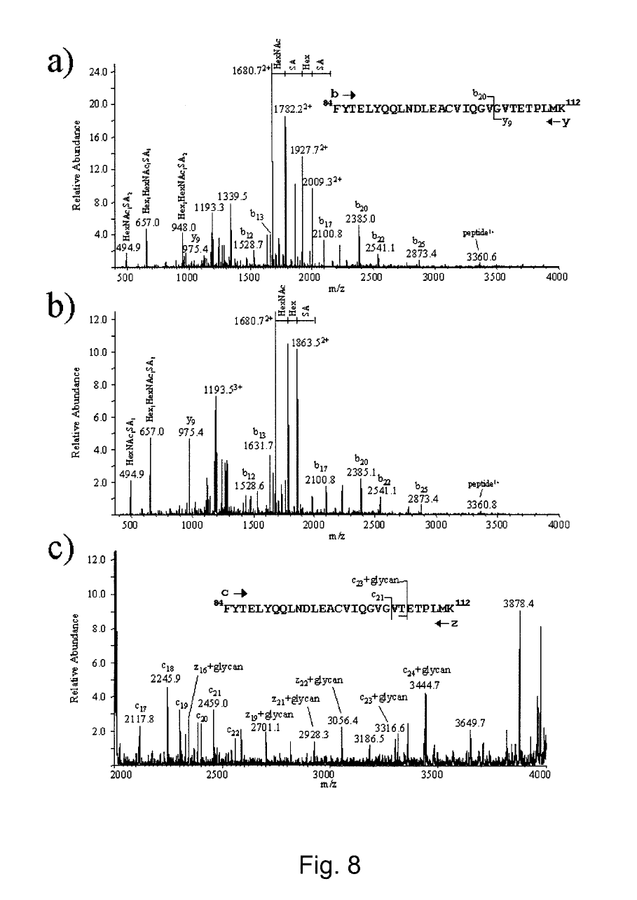

A detailed mass analysis and glycosylation pattern of the purified IFN-α2b was next performed by mass spectroscopy. An electrospray ionization (ESI) mass spectrum exhibiting the glycoform profiles associated with each charge state of purified IFN-α2b is shown (FIG. 7A). The masses of the principal glycoform of this protein correspond to the mature IFN-α2b peptide chain plus the glycans indicated (FIG. 7B). The most intense peak at 20,213 Da appears to be composed of the mature peptide chain plus a single core type-1 disialylated glycan (Hex1HexNAc1SA2). A MS/MS analysis of the tryptic glycopeptides confirms the composition of this glycan. The sialylated (mono and disialylated) glycoforms appear to constitute 75% of the total species. This percentage is likely to be underestimated, as some of the other peaks that cannot be assigned easily may be sialylated as well. The disialylated type 1 glycoform represents 50% of the total peak area while the monosialylated glycoform is 10% of the total. Using electron transfer dissociation, we also show that the glycan is linked to the expected threonine residue at position 106 (FIG. 8).

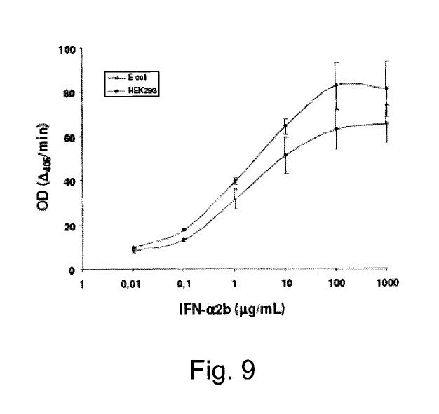

We next tested the purified glycosylated IFN-α2b produced in HEK293 for biological activity in comparison to non-glycosylated form produced in E. coli. Using a reporter gene assay we show that HEK-produced IFN-α2b is biologically active as it induces the production of a secreted alkaline phosphatase (SEAP) reporter enzyme under the control of the human ISG56 promoter (FIG. 9). In this assay, his assay shows that HEK-produced IFN-α2b is at least as active as bacterially produced IFN-α2b.)

### Discussion:

We describe here the generation of a HEK293 cell clone (D9) stably producing up to 316 mg of glycosylated human recombinant IFN-α2b per litre of serum-free culture in a simple fed batch culture maintained for only 7-8 days. This is the highest volumetric production of IFN-α2b reported for a mammalian system. In addition, IFN-α2b productivity of the D9 clone is stable for over 4 months without selection pressure. We have further developed a rapid and reliable method for the efficient recovery of biologically active IFN-α2b. We also provide an exhaustive analysis of its glycosylation, demonstrating by mass spectrometry that IFN-α2b produced in HEK293 cells is O-glycosylated and extensively sialylated. We show that the O-glycosylation of IFN-α2b produced in HEK293 cells is heterogeneous but similar to IFN-α2b produced by human peripheral blood leucocytes.

To date, the production of recombinant IFN-α2b and other cytokines in mammalian systems, particularly the development of clones stably expressing a cytokine of interest, has not been well exploited due to limitations in the volumetric productivity. One of the possible causes maybe that many cytokines exhibit strong anti-proliferative and cytotoxic activities on diverse cell lines [38,39], therefore strongly selecting for clones that show little or no cytokine expression. The D9 clone nonetheless grow almost as well as parental cells indicating that HEK293 cells can adapt to proliferate in the presence of high levels of IFN-α2b. This adaptability of HEK293 cells to a growth inhibitory cytokine suggests that they may be suitable hosts for the large-scale production of other interferons and cytokines. Clearly, a production capacity of >300 mg/L of IFN-α2b with more than 70% recovery and >98% purity is a strong argument in favour of using HEK293 cells for the large-scale productions of human recombinant cytokines. These results can be advantageously compared in terms of purified IFN-α2b obtained in E. colit (300 mg/L) [17] and the methyltrophic yeast Pichia pastoris [40], two hosts insensitive to the growth inhibitory activity of IFN-α2b. However the productivity reported by Srivastava et al [19] is 20-fold greater than that reported here for the D9 clone. Although we believe that the production capacity of HEK293 cells for IFN-α2b can be improved, we doubt that such productivity can ever be achieved in mammalian cells, at least for a cytokine.

However, the reported IFN-α2b recovery from prokaryotic systems ranges from 7.5-58% (Table 2), which is lower than what we were able to achieve (>75%). Purifications of recombinant proteins from prokaryotes usually require extraction from inclusion bodies and complicated refolding procedures, which reduce recovery yields [41]. Protein refolding is a critical step in the processing of biotherapeutics, as incompletely refolded species lower specific activity and may trigger an immune response. Antibodies to recombinant prokaryotic IFN-α2b have been detected in HCV patients with acquired resistance to IFN-α2b treatment [24,25], although it is not clear whether denatured IFN-α2b played a role in this case.

Because the vast majority of biotherapeutics including growth factors, cytokines and antibodies are secreted proteins, mammalian systems, unlike prokaryotes, allow for productions in perfusion as well as the development of non-denaturing purification procedures. The first and foremost advantage for the production of human recombinant proteins in mammalian systems is to generate proteins with the necessary posttranslational modifications required for full biological activity. N-glycosylation in particular, is often required for proper protein folding [42], protein-protein interactions, stability and optimal pharmacokinetics [43]. Although O-glycosylation is less critical for structure and function of proteins, it has been shown for example to increase the serum half-life of IGFBP6 by 2,3 folds over the non-glycosylated protein [44] and protect against proteolysis [45]. In a recent randomized study, O-glycosylated IFN-α2b was show to have an increased serum half life in comparison to non-glycosylated IFN-α2b [30]. We show here that human recombinant IFN-α2b produced in HEK293 cells is O-glycosylated and sialylated. Despite heterogeneity in the glycan structures, the nature and distribution of glycan moieties are quite similar to IFN-α2b naturally produced by human leukocytes [37]. Approximately 50% of our purified protein is disialylated, while another 30% is monosialylated in comparison to 50% and 10% respectively for leukocyte-produced IFN. Finally, we show that HEK-produced IFN-α2b is biologically active and is more potent than non-glycosylated E. coli-produced IFN.

The present invention demonstrates that the HEK293 cell line is a suitable host for the high volumetric production of glycosylated human recombinant IFN-α2b and potentially other cytokines.

### REFERENCES

### The Contents of the Entirety of Each of which are Incorporated by this Reference

1. Kaluz S, Kabat P, Gibadulinova A, Vojtassak J, Fuchsberger N, Kontsek P: Interferon alpha2b is the predominant subvariant detected in human genomic DNAs. Acta Virol 1994, 38: 101-104.
2. Mohanty S R, Kupfer S S, Khiani V: Treatment of chronic hepatitis B. Nat Clin Pract Gastroenterol Hepatol 2006, 3: 446-458.
3. Vogel W: Treatment of acute hepatitis C virus infection. J Hepatol 1999, 31 Suppl 1: 189-192.
4. Bajetta E, Del V M, Nova P, Fusi A, Daponte A, Sertoli M R et al.: Multicenter phase III randomized trial of polychemotherapy (CVD regimen) versus the same chemotherapy (CT) plus subcutaneous interleukin-2 and interferon-alpha2b in metastatic melanoma. Ann Oncol 2006, 17: 571-577.
5. Terheyden P, Becker J C, Kampgen E, Brocker E B: Sequential interferon-alpha2b, interleukin-2 and fotemustine for patients with metastatic melanoma. Melanoma Res 2000, 10: 475-482.
6. Eggermont A M, Suciu S, MacKie R, Ruka W, Testori A, Kruit W et al.: Postsurgery adjuvant therapy with intermediate doses of interferon alfa 2b versus observation in patients with stage IIb/III melanoma (EORTC 18952): randomised controlled trial. Lancet 2005, 366: 1189-1196.
7. Dezube B J: New therapies for the treatment of AIDS-related Kaposi sarcoma. Curr Opin Oncol 2000, 12: 445-449.
8. Angstreich G R, Smith B D, Jones R J: Treatment options for chronic myeloid leukemia: imatinib versus interferon versus allogeneic transplant. Curr Opin Oncol 2004, 16: 95-99.
9. Bonifazi F, de V A, Rosti G, Guilhot F, Guilhot J, Trabacchi E et al.: Chronic myeloid leukemia and interferon-alpha: a study of complete cytogenetic responders. Blood 2001, 98: 3074-3081.
10. Marler J J, Rubin J B, Trede N S, Connors S, Grier H, Upton J et al.: Successful antiangiogenic therapy of giant cell angioblastoma with interferon alfa 2b: report of 2 cases. Pediatrics 2002, 109: E37.
11. Pearlman B L: Hepatitis C treatment update. Am J Med 2004, 117: 344-352.
12. Manns M P, McHutchison J G, Gordon S C, Rustgi V K, Shiffman M, Reindollar R et al.: Peginterferon alfa-2b plus ribavirin compared with interferon alfa-2b plus ribavirin for initial treatment of chronic hepatitis C: a randomised trial. Lancet 2001, 358: 958-965.
13. Hoofnagle J H, Seeff L B: Peginterferon and ribavirin for chronic hepatitis C. N Engl J Med 2006, 355: 2444-2451.
14. Allain J P: Epidemiology of Hepatitis B virus and genotype. J Clin Virol 2006, 36 Suppl 1: S12-S17.
15. Seeff L B, Hoofnagle J H: Epidemiology of hepatocellular carcinoma in areas of low hepatitis B and hepatitis C endemicity. Oncogene 2006, 25: 3771-3777.
16. Picozzi V J, Kozarek R A, Traverso L W: Interferon-based adjuvant chemoradiation therapy after pancreaticoduodenectomy for pancreatic adenocarcinoma. Am J Surg 2003, 185: 476-480.
17. Babu K R, Swaminathan S, Marten S, Khanna N, Rinas U: Production of interferon-alpha in high cell density cultures of recombinant Escherichia coli and its single step purification from refolded inclusion body proteins. Appl Microbiol Biotechnol 2000, 53: 655-660.
18. Beldarrain A, Cruz Y, Cruz O, Navarro M, Gil M: Purification and conformational properties of a human interferon alpha2b produced in Escherichia coli. Biotechnol Appl Biochem 2001, 33: 173-182.
19. Srivastava P, Bhattacharaya P, Pandey G, Mukherjee K J: Overexpression and purification of recombinant human interferon alpha2b in Escherichia coli. Protein Expr Purif 2005, 41: 313-322.
20. Youngster S, Wang Y S, Grace M, Bausch J, Bordens R, Wyss D F: Structure, biology, and therapeutic implications of pegylated interferon alpha-2b. Curr Pharm Des 2002, 8: 2139-2157.
21. Glue P, Fang J W, Rouzier-Panis R, Raffanel C, Sabo R, Gupta S K et al.: Pegylated interferon-alpha2b: pharmacokinetics, pharmacodynamics, safety, and preliminary efficacy data. Hepatitis C Intervention Therapy Group. Clin Pharmacol Ther 2000, 68: 556-567.
22. Vyas K, Brassard D L, Delorenzo M M, Sun Y, Grace M J, Borden E C et al.: Biologic activity of polyethylene glycol12000-interferon-alpha2b compared with interferon-alpha2b: gene modulatory and antigrowth effects in tumor cells. J Immunother 2003, 26: 202-211.
23. Grace M J, Lee S, Bradshaw S, Chapman J, Spond J, Cox S et al.: Site of pegylation and polyethylene glycol molecule size attenuate interferon-alpha antiviral and antiproliferative activities through the JAK/STAT signaling pathway. J Biol Chem 2005, 280: 6327-6336.
24. Giannelli G, Antonelli G, Fera G, Del V S, Riva E, Broccia C et al.: Biological and clinical significance of neutralizing and binding antibodies to interferon-alpha (IFN-alpha) during therapy for chronic hepatitis C. Clin Exp Immunol 1994, 97: 4-9.
25. van der Eijk A A, Vrolijk J M, Haagmans B L: Antibodies neutralizing peginterferon alfa during retreatment of hepatitis C. N Engl J Med 2006, 354: 1323-1324.
26. Bianchi A A, McGrew J T: High-level expression of full-length antibodies using trans-complementing expression vectors. Biotechnol Bioeng 2003, 84: 439-444.
27. Grunberg J, Knogler K, Waibel R, Novak-Hofer I: High-yield production of recombinant antibody fragments in HEK-293 cells using sodium butyrate. Biotechniques 2003, 34: 968-972.
28. Wurm F M: Production of recombinant protein therapeutics in cultivated mammalian cells. Nat Biotechnol 2004, 22: 1393-1398.
29. Morser J: The effect of tunicamycin on interferon-alpha production in a human lymphoblastoid cell line (Namalwa). J Gen Virol 1982, 63 (Pt 1): 213-216.
30. Patel T B, Pequignot E, Parker S H, Leavitt M C, Greenberg H E, Kraft W K: Transgenic avian-derived recombinant human interferon-alpha2b (AVI-005) in healthy subjects: an open-label, single-dose, controlled study. Int J Clin Pharmacol Ther 2007, 45: 161-168.
31. Rapp J C, Harvey A J, Speksnijder G L, Hu W, Ivarie R: Biologically active human interferon alpha-2b produced in the egg white of transgenic hens. Transgenic Res 2003, 12: 569-575.
32. Sugyiama K, Ahorn H, Maurer-Fogy I, Voss T: Expression of human interferon-alpha 2 in Sf9 cells. Characterization of O-linked glycosylation and protein heterogeneities. Eur J Biochem 1993, 217: 921-927.
33. Zwarthoff E C, Bosveld I J, Vonk W P, Trapman J: Constitutive expression of a murine interferon alpha gene in hamster cells and characterization of its protein product. J Gen Virol 1985, 66 (Pt 4): 685-691.
34. Rossmann C, Sharp N, Allen G, Gewert D: Expression and purification of recombinant, glycosylated human interferon alpha 2b in murine myeloma NS0 cells. Protein Expr Purif 1996, 7: 335-342.
35. Durocher Y, Perret S, Kamen A: High-level and high-throughput recombinant protein production by transient transfection of suspension-growing human 293-EBNA1 cells. Nucleic Acids Res 2002, 30: E9.
36. Adolf G R, Kalsner I, Ahorn H, Maurer-Fogy I, Cantell K: Natural human interferon-alpha 2 is O-glycosylated. Biochem J 1991, 276 (Pt 2): 511-518.
37. Nyman T A, Kalkkinen N, Tolo H, Helin J: Structural characterisation of N-linked and O-linked oligosaccharides derived from interferon-alpha2b and interferon-alpha14c produced by Sendai-virus-induced human peripheral blood leukocytes. Eur J Biochem 1998, 253: 485-493.
38. Pokrovskaja K, Panaretakis T, Grander D: Alternative signaling pathways regulating type I interferon-induced apoptosis. J Interferon Cytokine Res 2005, 25: 799-810.
39. van Boxel-Dezaire A H, Rani M R, Stark G R: Complex modulation of cell type-specific signaling in response to type I interferons. Immunity 2006, 25: 361-372.
40. Shi L, Wang D, Chan W, Cheng L: Efficient expression and purification of human interferon alpha2b in the methylotrophic yeast, Pichia pastoris. Protein Expr Purif 2007, 54: 220-226.
41. Baneyx F, Mujacic M: Recombinant protein folding and misfolding in Escherichia coli. Nat Biotechnol 2004, 22: 1399-1408.
42. Molinari M: N-glycan structure dictates extension of protein folding or onset of disposal. Nat Chem Bio 12007, 3: 313-320.
43. Werner R G, Kopp K, Schlueter M: Glycosylation of therapeutic proteins in different production systems. Acta Paediatr Suppl 2007, 96: 17-22.
44. Marinaro J A, Casley D J, Bach L A: O-glycosylation delays the clearance of human IGF-binding protein-6 from the circulation. Eur J Endocrinol 2000, 142: 512-516.
45. Marinaro J A, Neumann G M, Russo V C, Leeding K S, Bach L A: O-glycosylation of insulin-like growth factor (IGF) binding protein-6 maintains high IGF-II binding affinity by decreasing binding to glycosaminoglycans and susceptibility to proteolysis. Eur J Biochem 2000, 267: 5378-5386.
46. Pham P L, Perret S, Cass B, Carpentier E, St-Laurent G, Bisson L et al.: Transient gene expression in HEK293 cells: peptone addition posttransfection improves recombinant protein synthesis. Biotechnol Bioeng 2005, 90: 332-344.
47. Coon J J, Ueberheide B, Syka J E, Dryhurst D D, Ausio J, Shabanowitz J et al.: Protein identification using sequential ion/ion reactions and tandem mass spectrometry. Proc Natl Acad Sci USA 2005, 102: 9463-9468.
48. Hogan J M, Pitteri S J, Chrisman P A, McLuckey S A: Complementary structural information from a tryptic N-linked glycopeptide via electron transfer ion/ion reactions and collision-induced dissociation. J Proteome Res 2005, 4: 628-632.
49. Syka J E, Coon J J, Schroeder M J, Shabanowitz J, Hunt D F: Peptide and protein sequence analysis by electron transfer dissociation mass spectrometry. Proc Natl Acad Sci USA 2004, 101: 9528-9533.
50. Durocher Y, Perret S, Thibaudeau E, Gaumond M H, Kamen A, Stocco R et al.: A reporter gene assay for high-throughput screening of G-protein-coupled receptors stably or transiently expressed in HEK293 EBNA cells grown in suspension culture. Anal Biochem 2000, 284: 316-326.
51. Pimienta E, Fando R, Sanchez J C, Vallin C: Secretion of human interferon alpha 2b by Streptomyces lividans. Appl Microbiol Biotechnol 2002, 58: 189-194.
52. Xu J, Tan L, Goodrum K J, Kieliszewski M J: High-yields and extended serum half-life of human interferon alpha2b expressed in tobacco cells as arabinogalactan-protein fusions. Biotechnol Bioeng 2007, 97: 997-1008.

Other advantages that are inherent to the structure are obvious to one skilled in the art. The embodiments are described herein illustratively and are not meant to limit the scope of the invention as claimed. Variations of the foregoing embodiments will be evident to a person of ordinary skill and are intended by the inventor to be encompassed by the following claims.

1. A method of purifying a recombinant interferon alpha2b (IFN-α2b) protein comprising:
(a) providing an aqueous mixture of the recombinant IFN-α2b protein and contaminating proteins, wherein the aqueous mixture comprises a culture medium separated from recombinant protein-producing mammalian cells;
(b) precipitating the contaminating proteins from the aqueous mixture by adjusting pH of the aqueous mixture to a pH in a range of from 0.5 to 6;
(c) separating the aqueous mixture from the precipitated contaminating proteins; and,
(d) eluting the separated aqueous mixture through a cation exchange column using a mobile phase with a salt or pH gradient, the gradient being from lower salt concentration or pH to higher salt concentration or pH, to produce a recombinant IFN-α2b interferon protein fraction separated from other components of the aqueous mixture.

2. The method of claim 1, wherein the interferon alpha2b is glycosylated.

3. The method of claim 1, wherein the contaminating proteins are precipitated at a pH in a range of 2 to 5.

4. The method of claim 1, wherein the contaminating proteins are precipitated at a pH in a range of 3.6 to 3.8.

5. The method of claim 1, wherein the medium is serum-free.

6. The method of claim 1, wherein the mammalian cells are HEK293 cells.

7. The method of claim 6, wherein the mammalian cells are HEK293-EBNA1 cells.

8. The method of claim 1, wherein the cells contain an expression plasmid comprising an EBV-oriP nucleotide sequence, a nucleotide sequence encoding α-interferon and a promoter for the nucleotide sequence encoding α-interferon.

9. The method of claim 8, wherein the expression plasmid comprises pYD7-IFNα2b.

10. The method of claim 1, wherein the mammalian cells are HEK293-EBNA1 clones designated D9 as deposited with the International Depositary Authority of Canada on Dec. 5, 2008 under accession no. 021208/03.

11. The method of claim 1, wherein a pH gradient is used in step (d) and the pH gradient is from 3.5 to 6.

12. The method of claim 1, wherein the cation exchange column comprises a stationary phase having sulfonic acid moieties.

13. The method of claim 1, further comprising desalting the recombinant interferon protein fraction.

14. A human embryonic kidney cell clone designated D9 having accession no. 021208/03 deposited on Dec. 5, 2008 at the International Depositary Authority of Canada.


SEQUENCE LISTING (1 sequence)

SEQ ID NO: 1 - protein, 188 aa, Homo sapiens
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE